(19) United States
(12) Patent Application Publication
(10) Pub. No.: US 2016/0178469 A1
(43) Pub. Date: Jun. 23, 2016

(54) CHARACTERIZATION OF AVIATION GASOLINE

(71) Applicants: Scott K. Berkhous, Mickleton, NJ (US); Roger G. Gaughan, Biltmore Lake, NC (US)

(72) Inventors: Scott K. Berkhous, Mickleton, NJ (US); Roger G. Gaughan, Biltmore Lake, NC (US)

(73) Assignee: ExxonMobil Research and Engineering Company, Annandale, NJ (US)

(21) Appl. No.: 14/956,465

(22) Filed: Dec. 2, 2015

Related U.S. Application Data
(60) Provisional application No. 62/092,899, filed on Dec. 17, 2014.

(51) Int. Cl. G01L 23/24; G01N 33/22

(52) U.S. Cl. CPC G01L 23/24; G01N 33/22

(57) ABSTRACT

Methods are provided for characterizing an aviation gasoline for use in a spark-ignited engine based on analysis methods traditionally used for compression-ignition fuels. For example, an aviation gasoline can be characterized based on a combustion delay for the aviation gasoline, a heat release rate for the aviation gasoline, or a combination thereof. Analyzing an aviation gasoline based on characteristics traditionally used for compression-ignition fuels can allow for distinction between types of aviation gasolines that may appear to be similar under conventional octane tests but that have substantially different performance characteristics.

### CROSS-REFERENCE TO RELATED APPLICATIONS

This application claims the benefit of provisional U.S. Ser. No. 62/092,899, filed Dec. 17, 2014, the entire contents of which are expressly incorporated by reference herein.

### FIELD OF THE INVENTION

This invention relates to methods for characterizing aviation fuels for use in spark ignited combustion engines.

### BACKGROUND OF THE INVENTION

Naphtha boiling range fuels that are used as aviation gasoline have traditionally been qualified in part according to ASTM D2700, ASTM D909, and/or D2699. ASTM D2700 provides a method for determining a motor octane number (MON) for a potential aviation gasoline. This can allow a potential aviation gasoline to be characterized according to typical aviation gasoline grades, such as 80/87, 91/98, or 100/130. D6227 provides a specification for UL82 and UL87 grades of unleaded aviation gasoline. Unfortunately, for the more recently developed unleaded versions of aviation gasoline per the specifications in ASTM D7547, it has been determined that ASTM D909 is not suitable for characterization of unleaded gasolines.

U.S. Patent Application Publication No. 2008/0213914 describes methods for using a constant volume combustion chamber to determine an octane number for a light distillate fuel based. The methods are based on the concept that a light distillate can have more than one combustion region, so a data point from pressure versus time data can be sampled from each combustion region. The method includes using a series expansion around the one or more data points obtained by monitoring pressure over time during combustion. The data point selected from each combustion region is described as being an ignition delay for the combustion region.

A paper presented at the 11^{th} International Conference on Stability, Handling, and Use of Liquid Fuels titled “Using a Constant Volume Combustion Chamber Analyzer for Predicting Derived Cetane Number of Aviation Turbine Fuels (IASH 2009, Prague, Czech Republic, Oct. 18-22, 2009) describes use of constant volume combustion chambers for characterization of cetane number for diesel powered aircraft. The constant volume combustion chamber was used to determine cetane rating for various fuels that correlate to results obtained from engine testing according to the method in ASTM D613.

### SUMMARY OF THE INVENTION

In an aspect, a method of characterizing a naphtha boiling range sample is provided, including injecting a naphtha boiling range sample having a final boiling point of about 170° C. or less into a constant volume combustion chamber at an injection pressure of at least about 10 barg (1 MPag), the constant volume combustion chamber containing an amount of oxygen corresponding to at least a stoichiometric amount relative to the fuel compounds in the naphtha boiling range sample; combusting the naphtha boiling range sample in the constant volume combustion chamber; measuring a pressure in the constant volume combustion chamber during a measurement time, the measurement time substantially including the combusting of the naphtha boiling range sample; and determining a combustion delay for the naphtha boiling range sample, the combustion delay being defined as a time from a start of the injecting of the naphtha boiling range sample to a time where a measured pressure in the constant volume combustion chamber is at least about 10% of a maximum pressure measured in the constant volume combustion chamber during the measurement time.

Optionally, the method can further include determining a curve characterizing a change in measured pressure during the measurement time; and determining a characteristic value for the change in measured pressure during the measurement time, the determined characteristic value optionally being a full-width half-maximum value.

In another aspect, a method of characterizing a naphtha boiling range sample is provided, including combusting a plurality of aviation gasoline samples in one or more first constant volume combustion chambers; measuring a pressure in the one or more first constant volume combustion chambers during a measurement time for each of the combusted aviation gas samples; determining a combustion delay window based on the measured pressures for the combusted aviation gas samples, a combustion delay being defined as a time from a start of the injecting of the naphtha boiling range sample to a time where a measured pressure in the constant volume combustion chamber is at least about 10% of a maximum pressure measured in the constant volume combustion chamber during the measurement time; injecting a naphtha boiling range sample having a final boiling point of about 170° C. or less into a second constant volume combustion chamber at an injection pressure of at least about 10 barg (1 MPag), the constant volume combustion chamber containing an amount of oxygen corresponding to at least a stoichiometric amount relative to the fuel compounds in the naphtha boiling range sample; combusting the naphtha boiling range sample in the second constant volume combustion chamber; measuring a pressure in the second constant volume combustion chamber during a measurement time, the measurement time substantially including the combusting of the naphtha boiling range sample; determining a combustion delay for the naphtha boiling range sample; and identifying the naphtha boiling range sample as being fit for purpose as an aviation gasoline based in part on the determined combustion delay for the naphtha boiling range sample being within the combustion delay window.

Optionally, the method can further include determining a curve characterizing a change in measured pressure during the measurement time for the plurality of aviation gasoline samples; determining a characteristic value window for the change in measured pressure during the measurement time for the plurality of aviation gasoline samples; determining a curve characterizing a change in measured pressure during the measurement time for the naphtha boiling range sample; determining a characteristic value for the change in measured pressure during the measurement time for the naphtha boiling range sample; and identifying the naphtha boiling range sample as being fit for purpose as an aviation gasoline based in part on the determined characteristic value for the naphtha boiling range sample being within the characteristic value window, the determined characteristic value optionally being a full-width half-maximum value.

### BRIEF DESCRIPTION OF THE FIGURES

FIG. 1 shows examples of pressure versus time curves from combustion of various potential fuels in a constant volume combustion chamber.

FIG. 2 shows examples of change in pressure versus time curves from combustion of various potential fuels in a constant volume combustion chamber.

### DETAILED DESCRIPTION OF THE EMBODIMENTS

### Overview

In various aspects, methods are provided for characterizing an aviation gasoline for use in a spark-ignited engine based on analysis methods traditionally used for compression-ignition fuels. For example, an aviation gasoline can be characterized based on a combustion delay for the aviation gasoline, a heat release rate for the aviation gasoline, or a combination thereof. Analyzing an aviation gasoline based on characteristics traditionally used for compression-ignition fuels can allow for distinction between new types of aviation gasolines that may appear to be similar under conventional octane tests but that have substantially different performance characteristics.

Internal combustion engines can typically be characterized as corresponding to one of two types of engines. In spark-ignited internal combustion engines, a mixture of fuel and air is compressed without causing ignition or combustion of the air/fuel mixture based just on compression. A spark is then introduced into the air fuel mixture to start combustion at a desired timing. Fuels for use in spark-ignited internal combustion engines are often characterized based on an octane rating, which is a measure of the ability of a fuel to resist combustion based solely on compression. The octane rating is valuable information for a spark-ignited engine, as the octane rating indicates what type of engine timings may be suitable for use with a given fuel. However, because octane rating is related to the resistance of a fuel to combustion, the octane rating does not directly provide insight into the properties of a fuel after combustion has started. Instead, octane rating directly provides only information regarding the ignition delay of a potential fuel.

The other typical type of engine is a compression ignition engine. In compression ignition, a mixture of air and fuel is provided into a cylinder which is compressed. When a sufficient amount of compression occurs, the mixture of air and fuel combusts. This combustion occurs without the need to introduce a separate spark to ignite the air/fuel mixture. A fuel for a compression ignition engine can be characterized based on a cetane number, which is a measure of how quickly a fuel will ignite. Cetane number is also primarily based on the start of ignition for a potential fuel, and therefore does not directly provide information regarding the properties of a fuel after combustion has started.

For traditional types of aviation gasoline, the composition of the aviation gasoline can be relatively similar to a standard type. Because the nature of the composition varied within a limited range, using an octane rating to characterize the performance of the aviation gasoline can provide acceptable results, as the overall compositional profile should not change greatly between potential fuels.

It would be advantageous to be able to expand the types of compositions suitable for use as aviation gasolines. In terms of octane rating, experience with gasoline blends for motor vehicles has shown that wide variety of compositions can lead to suitable octane ratings for automobiles. However, use of gasoline in an aviation setting imposes additional requirements, and simply characterizing a potential aviation gasoline based on octane rating may not provide sufficient information to determine the suitability as an aviation gasoline. For example, because octane number relates primarily to the ignition delay of an aviation gasoline, the octane number by itself does not describe additional features of the fuel, such as the length of time to reach at least about 10% (or at least about 30%, or at least about 50%, or at least about 70%, or at least about 90%) of the maximum combustion pressure during combustion of a fuel, or the rate of change over time of pressure within an engine cylinder during combustion. These features are not important for a conventional automobile motor fuel, but may be critical for the operation in an aviation context.

It has been determined that additional characterization of a potential aviation gasoline can be performed by using a constant volume combustion chamber to determine a combustion delay for a fuel. In this discussion, an ignition delay is defined to have the traditional definition of the period of time between the start of injection of a fuel and the first identifiable pressure increase in the pressure versus time curve. In this discussion, a combustion delay is defined as the time required to achieve a specified percentage of the maximum chamber pressure during combustion. In various aspects, the specified percentage can be at least about 10% (for example at least about 30%, at least about 50%, at least about 70%, or at least about 90%) of the maximum chamber pressure. This can be determined based on measurement of the pressure in a constant volume combustion chamber versus time. Based on the definitions for ignition delay and combustion delay, it is clear that these delays can provide different types of information. The ignition delay corresponds to the traditional data point used to determine an octane number or cetane rating. Because the ignition delay is based on the start of the increase in pressure, the ignition delay (and therefore the octane number or cetane rating) can provide little or no additional information regarding the nature of combustion once ignition has occurred. By contrast, the combustion delay can be dependent on both the time for the start of ignition as well as the time required to reach the maximum pressure once ignition has started. By using combustion delay rather than ignition delay to characterize an aviation gasoline, additional distinctions can be made between various types of aviation gasolines that appear to have similar octane numbers. This can allow, for example, for improved characterization of potential aviation gasolines that might have non-traditional compositions.

Additionally or alternately, a potential aviation gasoline can be characterized based on the rate of pressure change during combustion. The rate of pressure change during combustion roughly correlates with the rate of heat release during combustion, and therefore the rate of pressure change can be used as an indicator of the combustion rate for a fuel. Although the rate of pressure change is related to the combustion delay, it can also provide additional information. The combustion delay for a fuel is dependent in part on the amount of time needed for combustion to begin, which roughly corresponds to the traditional ignition delay. By contrast, the width of the curve for the rate of pressure change can provide a description of how quickly (or slowly) combustion can occur once ignition has started. This separate method for characterizing a fuel can further distinguish between fuels that might otherwise appear suitable for use. For example, a fuel with a sufficient delay in initial combustion may appear to have a suitable octane rating. However, if the rate of combustion after ignition is too slow, the fuel may be a poor choice for use as an aviation gasoline.

### Characterization of Combustion Delay and Rate of Pressure Change

In various embodiments, the combustion delay and/or the rate of pressure change during combustion for an aviation gasoline can be characterized using a constant volume combustion chamber. The characterization can be performed at any convenient temperature that allows for consistent characterization. For example, a suitable temperature in the combustion chamber can be a temperature between about 450° C. and 650° C., such as a temperature of about 575° C.

With regard to pressure, the characterization can be performed at an elevated pressure relative to conventional techniques for determining an octane number for an aviation gasoline. For example, prior to combustion, the pressure within the constant volume chamber can be from about 5.0 barg (˜0.5 MPag) to about 25.0 barg (˜2.5 MPag), for example from about 5.0 barg (˜0.5 MPag) to about 22 barg (˜2.2 MPag), from about 10.0 barg (˜1.0 MPag) to about 22.0 barg (˜2.2 MPag), or from about 10.0 barg (˜1.0 MPag) to about 20 barg (˜2.0 MPag). Operating at an elevated pressure can also allow for injection of fuel into the constant volume chamber at elevated pressure. The fuel can be injected at the pressure in the chamber, or the fuel can be injected at an initial pressure that is greater than the chamber pressure. In some embodiments, the fuel can be injected at a pressure of about 400 barg (˜40 MPag) to about 2000 barg (˜200 MPag). For example, the fuel can be injected at a pressure of at least about 400 barg (˜40 MPag), at least about 500 barg (˜50 MPag), or at least about 600 barg (˜60 MPag). Additionally or alternately, the fuel can be injected at a pressure of about 2000 barg (˜200 MPag) or less, for example about 1500 barg (˜150 MPag) or less or about 1200 barg (˜120 MPag) or less. The elevated pressure in the chamber and/or elevated pressure for injection of the fuel can assist with mixing of the fuel with the air in the chamber. This improved mixing can allow for increased differentiation between the characteristics of various potential aviation gasoline compositions.

The method for injecting the fuel into the constant volume chamber can be any convenient method. Optionally, the method for injecting the fuel into the constant volume chamber can include use of an injector suitable for injecting diesel fuel into a chamber, such as an injector comprising a plurality of orifices for producing jets of injected fuel and/or a plurality of diesel fuel injectors. This can be in contrast to the typical injector used for characterization of a gasoline octane number, which can correspond to a pintle type injector (small needle with single hole).

An example of a suitable apparatus for performing a constant volume combustion chamber characterization of an aviation gasoline is a Cetane ID 510 constant volume combustion chamber, available from PAC, LP of Houston, Tex.

Based on the conditions in a constant volume combustion chamber, a combustion delay and/or a rate of pressure change versus time can be determined for potential aviation gasoline compositions. During a test of a potential aviation gasoline, a combustion chamber can be charged with air at a specified pressure. The air in the chamber can then be heated to a desired set point temperature for the test. The chamber can be held at a substantially constant temperature/constant pressure at that point until fuel is introduced into the chamber. Fuel can then be injected into the chamber for a predetermined amount of time, such as an amount of time that corresponds to a desired amount of fuel for injection. An analyzer can measure pressure as function of time after injection of the fuel. Combustion could start during injection, but typically combustion does not start until after completing the injection of the fuel. After injection begins, an initial slight drop in pressure may be observed due to cooling as injected fuel evaporates. A “cool ignition” period can then start, which can correspond to a minor rise in pressure. The end of this “cool ignition” period can correspond to the ignition delay, which can be defined (such as in ASTM D7668) as the time to achieve an increase of more than ˜0.02 bar over the initial set point chamber pressure (D7668). By contrast, the combustion delay can be defined based on the time required to achieve a percentage of the maximum chamber pressure during combustion, such as about 10% of the maximum chamber pressure, about 30% of the maximum chamber pressure, or about 50% of the maximum chamber pressure.

As combustion occurs, the pressure in the constant volume can be monitored in any convenient unit of time that allows for characterization of the pressure on a millisecond scale. This can allow a curve of pressure versus time to be developed. The combustion delay can then be calculated based on the time difference between the start of injection of fuel until the pressure in the constant volume reaches a specified amount (such as 10% or 30% or 50%) of the maximum measured pressure. It is noted that a diesel fuel can typically have a combustion delay at ˜50% of maximum measured pressure of at least about 5 ms to about 15 ms. A suitable aviation gas can have a combustion delay at ˜50% of maximum measured pressure of about 20 ms to about 170 ms.

In addition to determining combustion delay, the pressure versus time curve can also be used to determine the change in pressure versus time. This can be determined in any convenient manner using the collected data, such as by fitting a functional form and taking a derivative, calculating actual change values between discrete data points (optionally with smoothing of the data), or combinations thereof.

One option for using a constant volume combustion chamber for characterization of aviation gasoline compositions can be to construct a library of known compositions suitable for use as aviation gasoline, and then use the combustion delays and/or change in pressure versus time values to determine an envelope for fuels with behavior that may be similar to known, tested fuels. Potential aviation gasoline compositions with combustion delays and/or pressure change versus time values within the threshold ranges defined from the library of known values can be identified as suitable for use, while potential aviation gasoline compositions with values outside of the threshold ranges can be identified as requiring further characterization prior to use.

In certain embodiments, a potential fuel composition can be identified as suitable and/or fit for purpose based on the potential fuel composition having a combustion delay and/or a pressure change versus time value within a defined performance window. For example, a fuel composition can be identified as suitable and/or fit for purpose based on having a combustion delay at ˜10% and/or ˜30% and/or ˜50% of maximum measured pressure of about 20 ms to 200 ms, for example about 20 ms to about 160 ms, about 20 ms to about 150 ms, about 50 ms to about 200 ms, about 50 ms to about 160 ms, about 50 ms to about 150 ms, about 80 ms to about 200 ms, about 80 ms to about 160 ms, about 80 ms to about 150 ms, about 90 ms to about 200 ms, about 90 ms to about 160 ms, or about 90 ms to about 150 ms. Additionally or alternatively, a fuel composition can be identified as suitable and/or fit for purpose based on having a pressure change versus time curve (which can be correlated with heat release) where the full-width half-maximum value for the curve is about 4.0 ms to about 12.0 ms, for example about 4.5 ms to about 12.0 ms, about 5.0 ms to about 12.0 ms, about 5.5 ms to about 12.0 ms, about 4.0 ms to about 11.0 ms, about 4.5 ms to about 11.0 ms, about 5.0 ms to about 11.0 ms, about 5.5 ms to about 11.0 ms, about 4.0 ms to about 10.0 ms, about 4.5 ms to about 10.0 ms, about 5.0 ms to about 10.0 ms, or about 5.5 ms to about 10.0 ms.

### Aviation Gasoline Characteristics

In some embodiments, the compositions characterized using a constant volume combustion chamber can be compositions that (other than combustion delay and/or rate of burning) have suitable properties for use as an aviation gasoline. For example, some compositions can have a density of at least about 6.01 lb/gal at ˜15° C. (about 0.721 kg/l), for example from about 0.72 kg/l at ˜15° C. to about 0.83 kg/l. Additionally or alternately, the compositions can have an emission coefficient of ˜18.355 pounds CO₂ per gallon (˜2.1994 kg/l). Further additionally or alternately, the compositions can have a vapor pressure from about 5.5 psi (about 40 kPa) to about 7.0 psi (about 50 kPa), for example from about 38 kPa to about 62 kPa. Still further additionally or alternately, about 10 vol % to about 40 vol % of the fuel can having a boiling point of less than about 167° F. (about 75° C.), about 90 vol % of the fuel can have a boiling point of about 275° F. (about 135° C.) or less, and/or a final boiling point of the composition can be about 338° F. (about 170° C.) or less. Yet further additionally or alternately, a sum of the temperatures for evaporation of 10 vol % and 50 vol % of the aviation gasoline can be at least about 135° C., and/or, for octanes below about 90, the sum can be at least about 150° C. Yet again further additionally or alternately, the freezing point for an aviation gasoline can be about −58° C. or less.

The fuel compositions described herein can generally be referred to as naphtha boiling range compositions, such as naphtha boiling range compositions with a final boiling point of about 170° C. or less. The naphtha boiling range applicable for use in avgas is defined herein as being about 36° C. (roughly the boiling point of n-pentane) to about 170° C.

Traditionally, a tool for cetane characterization can be designed for characterization of fuel compositions that have a cetane number of at least about 39.4. In various embodiments, the aviation gasoline compositions described herein can be suitable for use in spark ignition combustion engines. In such embodiments, the aviation gasoline compositions can have cetane numbers of about 39.0 or less, for example about 37.0 or less or about 35.0 or less. It is noted that, for cetane numbers below about 30, the equations for deriving the cetane number are outside of the qualified range for the equations. Thus, although a cetane value of 30 or less can theoretically be calculated, cetane values below 30 are not typically reported. The octane numbers (MON) of the aviation gasoline compositions can be at least about 80 octane, for example at least about 90 octane, at least about 95 octane, or at least about 100, and optionally up to a performance number of about 200 octane. It is noted that motor octane numbers above 100 are outside of the typical definition for motor octane. For situations where an apparent motor octane number would be greater than 100, the number can be more properly referred to as a performance number.

### Additional Embodiments

The methods according to the invention can further include one or more embodiments listed below/herein.

Embodiment 1. A method of characterizing a naphtha boiling range sample, comprising: injecting a naphtha boiling range sample having a final boiling point of about 170° C. or less into a constant volume combustion chamber at an injection pressure of at least about 10 barg (1 MPag), the constant volume combustion chamber containing an amount of oxygen corresponding to at least a stoichiometric amount relative to the fuel compounds in the naphtha boiling range sample; combusting the naphtha boiling range sample in the constant volume combustion chamber; measuring a pressure in the constant volume combustion chamber during a measurement time, the measurement time substantially including the combusting of the naphtha boiling range sample; and determining a combustion delay for the naphtha boiling range sample, the combustion delay being defined as a time from a start of the injecting of the naphtha boiling range sample to a time where a measured pressure in the constant volume combustion chamber is at least about 10% of a maximum pressure measured in the constant volume combustion chamber during the measurement time.

Embodiment 2. The method of Embodiment 1, the method further comprising: determining a curve characterizing a change in measured pressure during the measurement time; and determining a characteristic value for the change in measured pressure during the measurement time, the determined characteristic value optionally being a full-width half-maximum value.

Embodiment 3. A method of characterizing a naphtha boiling range sample, comprising: combusting a plurality of aviation gasoline samples in one or more first constant volume combustion chambers; measuring a pressure in the one or more first constant volume combustion chambers during a measurement time for each of the combusted aviation gas samples; determining a combustion delay window based on the measured pressures for the combusted aviation gas samples, a combustion delay being defined as a time from a start of the injecting of the naphtha boiling range sample to a time where a measured pressure in the constant volume combustion chamber is at least about 10% of a maximum pressure measured in the constant volume combustion chamber during the measurement time; injecting a naphtha boiling range sample having a final boiling point of about 170° C. or less into a second constant volume combustion chamber at an injection pressure of at least about 10 barg (1 MPag), the constant volume combustion chamber containing an amount of oxygen corresponding to at least a stoichiometric amount relative to the fuel compounds in the naphtha boiling range sample; combusting the naphtha boiling range sample in the second constant volume combustion chamber; measuring a pressure in the second constant volume combustion chamber during a measurement time, the measurement time substantially including the combusting of the naphtha boiling range sample; determining a combustion delay for the naphtha boiling range sample; and identifying the naphtha boiling range sample as being fit for purpose as an aviation gasoline based in part on the determined combustion delay for the naphtha boiling range sample being within the combustion delay window.

Embodiment 4. The method of Embodiment 3, the method further comprising: determining a curve characterizing a change in measured pressure during the measurement time for the plurality of aviation gasoline samples; determining a characteristic value window for the change in measured pressure during the measurement time for the plurality of aviation gasoline samples; determining a curve characterizing a change in measured pressure during the measurement time for the naphtha boiling range sample; determining a characteristic value for the change in measured pressure during the measurement time for the naphtha boiling range sample; and identifying the naphtha boiling range sample as being fit for purpose as an aviation gasoline based in part on the determined characteristic value for the naphtha boiling range sample being within the characteristic value window, the determined characteristic value optionally being a full-width half-maximum value.

Embodiment 5. The method of Embodiment 3 or 4, wherein the second constant volume combustion chamber is selected from the one or more first constant volume combustion chambers.

Embodiment 6. The method of any of the above embodiments, wherein the determined combustion delay is from about 20 ms to about 200 ms, for example from about 80 ms to about 160 ms, the combustion delay window is from about 20 ms to about 200 ms (for example from about 80 ms to about 160 ms), or a combination thereof.

Embodiment 7. The method of any of Embodiments 2 or 4-6, wherein the determined characteristic value is from about 4.0 ms to about 12 ms (for example from about 4.5 ms to about 11 ms), the characteristic value window is from about 4.0 ms to about 12 ms (for example from about 4.5 ms to about 11 ms), or a combination thereof.

Embodiment 8. The method of any of the above embodiments, wherein the naphtha boiling range sample has a cetane number of about 39 or less (for example about 37 or less, about 35 or less, or about 30 or less), and/or wherein the naphtha boiling range sample has an octane number (MON) of at least about 80 (for example at least about 90 or at least about 95).

Embodiment 10. The method of any of the above embodiments, wherein the naphtha boiling range sample is injected into the constant volume combustion chamber using an injector comprising a plurality of orifices.

Embodiment 11. The method of any of the above embodiments, wherein the combustion delay is defined as a time from a start of the injecting of the naphtha boiling range sample to a time where a measured pressure in the constant volume combustion chamber is at least about 30% of a maximum pressure, or at least about 50% of a maximum pressure, measured in the constant volume combustion chamber during the measurement time.

Embodiment 12. The method of any of the above embodiments, wherein the injection pressure is from about 400 barg (40 MPag) to about 2000 barg (200 MPag), for example from about 500 barg (about 50 MPag) to about 2000 barg (about 200 MPag), from about 400 barg (about 40 MPag) to about 1500 barg (about 150 MPag), or from about 500 barg (50 MPag) to about 1500 barg (150 MPag).

Embodiment 13. The method of any of the above embodiments, wherein the naphtha boiling range sample is combusted without use of a spark for ignition.

Example of Characterization using Constant Volume Combustion Chamber

The following example describes characterizations that were performed on a series of potential aviation gasoline compositions using a constant volume combustion chamber.

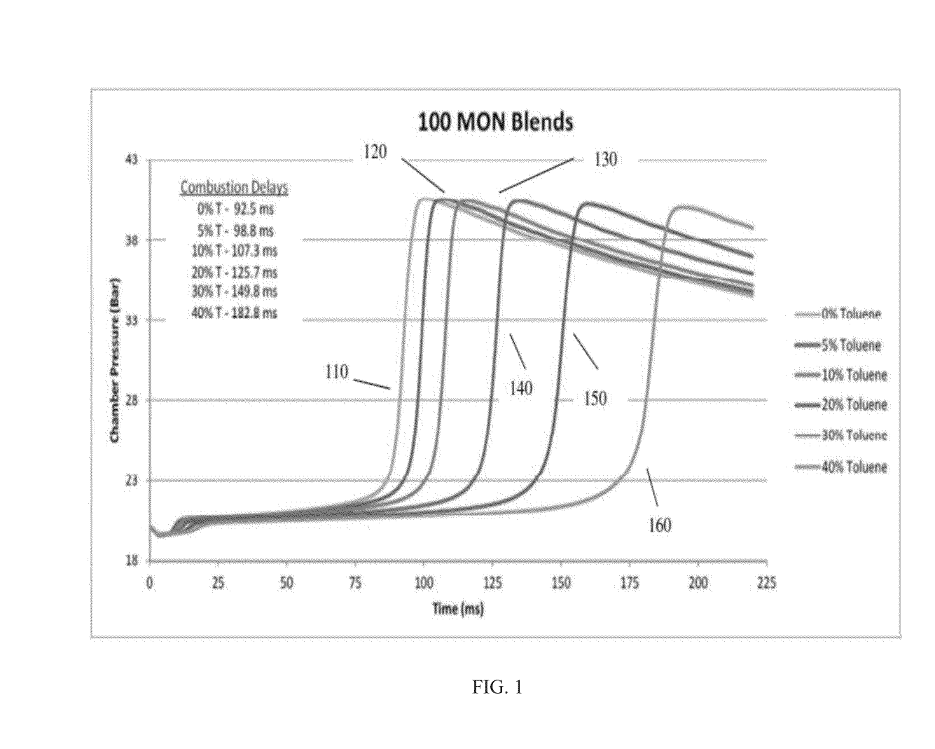

A series of fuel blends using primary reference fluids (Isooctane, Heptane, and Toluene) were made with the intent of keeping the nominal Motor Octane Number (MON) at a roughly constant value, while varying the content of toluene between ˜0% vol and ˜40 vol %. The various blends were analyzed using a CID 510 constant volume combustion chamber (CVCC) instrument. Analysis of these blends in the CID510 CVCC instrument appeared to show dramatic differences in the combustion performance of the blends as shown in the data below. FIG. 1 shows the pressure versus time profiles for the various blends. In FIG. 1, the pressure versus time profiles correspond to a profile for toluene of ˜0 vol % (110), ˜5 vol % (120), ˜10 vol % (130), ˜20 vol % (140), ˜30 vol % (150), and ˜40 vol % (160) As shown in FIG. 1, increasing the amount of toluene in a fuel blend appeared to result in longer combustion delays. At toluene amounts of ˜10 vol % or less (profiles 110, 120, and 130), the combustion delay (time required to achieve ˜50% of maximum pressure) appeared to be less than about 120 ms. Further addition of toluene appeared to result in longer delays, with a ˜40 vol % toluene feed (profile 160) having a combustion delay (˜50% of maximum pressure) of greater than about 180 ms. Thus, as shown in FIG. 1, the combustion delay varied by greater than 95% (about 90 ms to about 180 ms) between samples containing the minimum and maximum amounts of toluene.

As a comparison, samples of the fuel compositions containing ˜10 vol % toluene, ˜20 vol % toluene, and ˜40 vol % toluene were tested for motor octane number (MON) according to the method in ASTM D2700. Table 1 shows the results from characterizing the fuel compositions. As shown in Table 1, each of the fuel compositions had a MON value of between about 94 and about 97, which are suitable values for some grades of aviation gas. As shown in Table 1, the difference in MON between the fuel compositions with ˜10 vol % and ˜20 vol % toluene appeared to be within the reproducibility of the ASTM D2700 test method. By contrast, the combustion delays for the compositions having ˜10 vol % and ˜20 vol % toluene were about 107 ms and about 126 ms, respectively. These values can be readily distinguished using the CVCC test method. The differences between the fuel compositions having ˜10 vol % and ˜40 vol % toluene appeared to be even more stark. Using the ASTM D2700 method, Table 1 shows that only about a 2% change was measured between the ˜10 vol % toluene and ˜40 vol % toluene samples. This is in contrast to an about 70% increase in combustion delay (about 107 ms versus about 183 ms) as measured using the CVCC test method. This second comparison is particularly notable, as the D2700 MON values for the ˜10 vol % and ˜40 vol % toluene compositions appeared to indicate that both might be suitable for use as a 91/98 type aviation gasoline composition. However, based on the combustion delay, the composition containing ˜40 vol % toluene appeared to have a combustion delay that is longer than would be expected for a traditional aviation gasoline composition. Use of combustion delay to characterize the fuel compositions can allow the ˜40 vol % toluene composition to be identified as outside of normal aviation gasoline standards, so that additional characterization might be required prior to approving the composition for use as an aviation gasoline.

TABLE 1
ASTM D2700 MON Determination of
100 MON Nominal Blended Samples

| Vol % | D2700 | D2700 | D2700 | Average |
|---|---|---|---|---|
| Toluene | MON #1 | MON #2 | MON #3 | MON |
| ~10 | ~97.6 | | | ~97.6 |
| ~20 | ~96.1 | ~96.0 | | ~96.1 |
| ~40 | ~95.1 | ~95.5 | ~95.2 | ~95.3 |

Based on the data shown in FIG. 1 relative to the data in Table 1, use of a CVCC type instrument can allow for characterization of potential aviation gasoline compositions in a manner that has increased sensitivity to compositional changes (e.g., aromatic content) than a conventional characterization according to ASTM D2700. As a result, the CVCC type characterization can be used to identify an operational window or envelope of combustion performance range, such as an operation window based on traditional aviation gasoline compositions. By defining this range of acceptable combustion delay values, new fuel compositions that may have reduced or minimized similarity to conventional compositions can be assessed for acceptable combustion performance. If a proposed fuel composition falls outside of the defined combustion performance range, the proposed fuel composition can be identified as needing further testing prior to being certified as fit for purpose for use as an aviation gasoline.

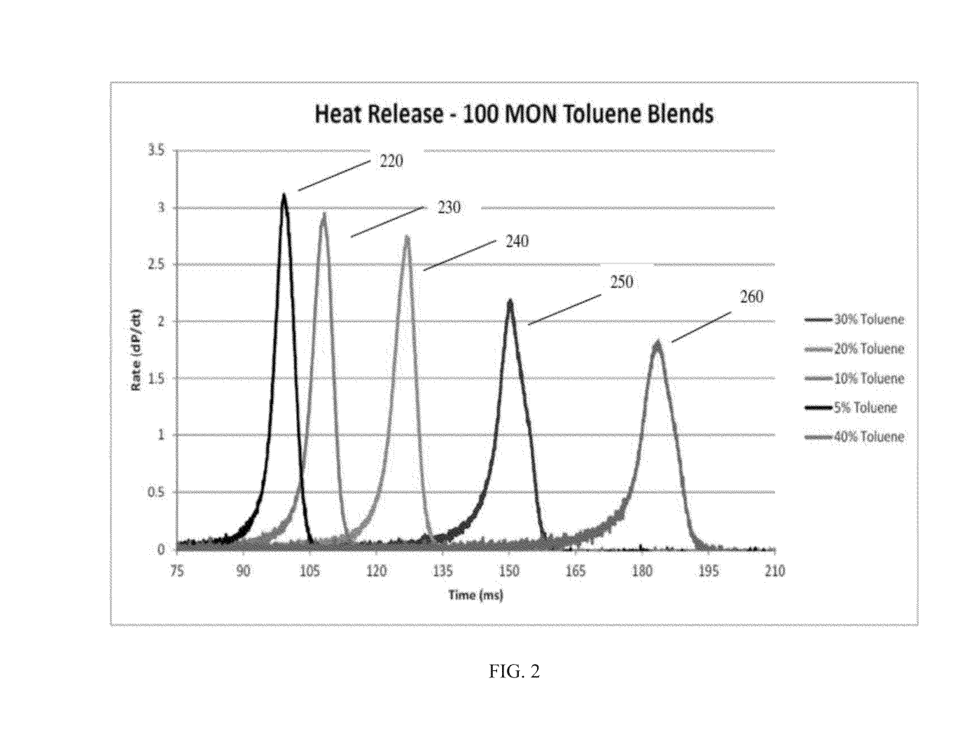

The combustion profile shown in FIG. 1 can be used to further generate information for characterizing potential aviation gasoline compositions. FIG. 2 shows the rate of pressure change as a function of time for the combustion data shown in FIG. 1. The profiles in FIG. 2 correspond to toluene contents of ˜5 vol % (220), ˜10 vol % (230), ˜20 vol % (240), ˜30 vol % (250), and ˜40 vol % (260). The data in FIG. 2 can be correlated with the rate of heat release by a potential fuel composition during combustion. The data shown in FIG. 2 can provide information related to the combustion performance and potential power generated in a piston aircraft engine. As shown in FIGS. 1 and 2, increasing the amount of aromatics (represented by % toluene) can apparently have at least one of two potential major impacts on heat release rate. First, increasing toluene concentration can delay the onset of combustion and thus the heat release. Secondly, increasing toluene concentration can decrease the intensity or rate of the heat release. This can impact the amount of power produced by an engine.

The heat release and/or change in pressure versus time data shown in FIG. 2 can also be used for characterizing a fuel composition as suitable/fit for purpose, either alone or in combination with the combustion delay as determined from the type of data shown in FIG. 1. For example, the data shown in FIG. 2 can be used to characterize the width of the pressure change curve, such as to possibly calculate a full-width half-maximum value for the pressure change (heat release) curve. As for combustion delay, such full-width half-maximum values can be determined for conventional aviation gasoline compositions to develop a combustion performance window or envelope that is deemed suitable and/or fit for purpose. New compositions can then be compared with the performance window for heat release, so that compositions that produce values outside of the performance window are identified for further testing prior to being determined as suitable and/or fit for purpose. It is noted that the use of a full-width half-maximum characterization is described herein only as an example. More generally, any other convenient statistical measure for characterizing the shape of a curve, such as representing a curve as one or more Gaussian shaped curves, can be used to develop a performance window standard.

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth herein but rather that the claims be construed as encompassing all the features of patentable novelty which reside in the present invention, including all features which would be treated as equivalents thereof by those skilled in the art to which the invention pertains.

What is claimed is:

1. A method of characterizing a naphtha boiling range sample, comprising:
injecting a naphtha boiling range sample having a final boiling point of about 170° C. or less into a constant volume combustion chamber at an injection pressure of at least about 10 barg (about 1 MPag), the constant volume combustion chamber containing an amount of oxygen corresponding to at least a stoichiometric amount relative to the fuel compounds in the naphtha boiling range sample;
combusting the naphtha boiling range sample in the constant volume combustion chamber;
measuring a pressure in the constant volume combustion chamber during a measurement time, the measurement time substantially including the combusting of the naphtha boiling range sample; and
determining a combustion delay for the naphtha boiling range sample, the combustion delay being defined as a time from a start of the injecting of the naphtha boiling range sample to a time where a measured pressure in the constant volume combustion chamber is at least about 10% of a maximum pressure measured in the constant volume combustion chamber during the measurement time.

2. The method of claim 1, wherein the combustion delay is about 20 ms to about 200 ms.

3. The method of claim 1, the method further comprising:
determining a curve characterizing a change in measured pressure during the measurement time; and
determining a characteristic value for the change in measured pressure during the measurement time.

4. The method of claim 3, wherein the determined characteristic value is a full-width half-maximum value.

5. The method of claim 4, wherein the determined characteristic value is from about 4.0 ms to about 12 ms.

6. The method of claim 1, wherein the naphtha boiling range sample has a cetane number of about 30 or less, an octane number (MON) of at least about 80, or a combination thereof.

7. The method of claim 1, wherein the naphtha boiling range sample is injected into the constant volume combustion chamber using an injector comprising a plurality of orifices.

8. The method of claim 1, wherein the combustion delay is defined as a time from a start of the injecting of the naphtha boiling range sample to a time where a measured pressure in the constant volume combustion chamber is at least about 50% of a maximum pressure measured in the constant volume combustion chamber during the measurement time.

9. The method of claim 1, wherein the injection pressure is from about 400 barg (about 40 MPag) to about 2000 barg (about 200 MPag).

10. The method of claim 1, wherein the naphtha boiling range sample is combusted without use of a spark for ignition.

11. A method of characterizing a naphtha boiling range sample, comprising:
combusting a plurality of aviation gasoline samples in one or more first constant volume combustion chambers;
measuring a pressure in the one or more first constant volume combustion chambers during a measurement time for each of the combusted aviation gas samples;
determining a combustion delay window based on the measured pressures for the combusted aviation gas samples, a combustion delay being defined as a time from a start of the injecting of the naphtha boiling range sample to a time where a measured pressure in the constant volume combustion chamber is at least about 10% of a maximum pressure measured in the constant volume combustion chamber during the measurement time;
injecting a naphtha boiling range sample having a final boiling point of about 170° C. or less into a second constant volume combustion chamber at an injection pressure of at least about 10 barg (about 1 MPag), the constant volume combustion chamber containing an amount of oxygen corresponding to at least a stoichiometric amount relative to the fuel compounds in the naphtha boiling range sample;
combusting the naphtha boiling range sample in the second constant volume combustion chamber;
measuring a pressure in the second constant volume combustion chamber during a measurement time, the measurement time substantially including the combusting of the naphtha boiling range sample;
determining a combustion delay for the naphtha boiling range sample; and
identifying the naphtha boiling range sample as being fit for purpose as an aviation gasoline based in part on the determined combustion delay for the naphtha boiling range sample being within the combustion delay window.

12. The method of claim 11, wherein the combustion delay window is from about 20 ms to about 200 ms.

13. The method of claim 11, wherein the determined combustion delay is from about 80 ms to about 160 ms.

14. The method of claim 11, the method further comprising:
determining a curve characterizing a change in measured pressure during the measurement time for the plurality of aviation gasoline samples;
determining a characteristic value window for the change in measured pressure during the measurement time for the plurality of aviation gasoline samples;
determining a curve characterizing a change in measured pressure during the measurement time for the naphtha boiling range sample;
determining a characteristic value for the change in measured pressure during the measurement time for the naphtha boiling range sample; and
identifying the naphtha boiling range sample as being fit for purpose as an aviation gasoline based in part on the determined characteristic value for the naphtha boiling range sample being within the characteristic value window.

15. The method of claim 14, wherein the determined characteristic value is a full-width half-maximum value.

16. The method of claim 14, wherein the characteristic value window is from about 4.0 ms to about 12 ms.

17. The method of claim 14, wherein the determined characteristic value is from about 4.5 ms to about 11 ms.

18. The method of claim 11, wherein the second constant volume combustion chamber is selected from the one or more first constant volume combustion chambers.

19. The method of claim 11, wherein the combustion delay being defined as a time from a start of the injecting of the naphtha boiling range sample to a time where a measured pressure in the constant volume combustion chamber is at least about 30% of a maximum pressure measured in the constant volume combustion chamber during the measurement time.

20. The method of claim 11, wherein the naphtha boiling range sample has a cetane number of about 30 or less, an octane number (MON) of at least about 80, or a combination thereof.
